(19) United States
(12) Patent Application Publication
(10) Pub. No.: US 2014/0316220 A1
(43) Pub. Date: Oct. 23, 2014

(54) Personal Health Monitoring System

(71) Applicant: Michael L. Sheldon, Georgetown, TX (US)

(72) Inventor: Michael L. Sheldon, Georgetown, TX (US)

(21) Appl. No.: 14/321,352

(22) Filed: Jul. 1, 2014

Related U.S. Application Data
(63) Continuation-in-part of application No. 14/156,582, filed on Jan. 16, 2014.
Application No. 14/156,582 is a continuation-in-part of application No. 13/908,661, filed on Jun. 3, 2013.
(60) Provisional application No. 61/850,507, filed on Feb. 15, 2013.
(60) Provisional application No. 61/850,507, filed on Feb. 15, 2013.

(51) Int. Cl. A61B 5/00; G06F 19/00

(52) U.S. Cl. CPC A61B 5/7275; G06F 19/3437; G06F 19/3406
USPC 600/301

(57) ABSTRACT

A personal health monitor device includes a memory for collecting and storing attributes from an individual and a processor for quantizing each attribute in such a way as to indicate a normal range for that attribute and for measuring deviations from that normal range. The processor further calculates the well-being of the individual using the deviations measured. The results are displayed indicating the well-being of the individual.

### CROSS-REFERENCE TO RELATED APPLICATION

This application is a continuation-in-part of U.S. patent application Ser. No. 14/156,582, entitled “Personal Health Monitoring System,” filed Jan. 16, 2014, and is a continuation-in-part of U.S. patent application Ser. No. 13/908,661, entitled “Personal Health Monitoring System,” filed Jun. 3, 2013, both of which claim the benefit under 35 U.S.C. §119(e) of U.S. Provisional Application Ser. No. 61/850,507, entitled “Personal Health Monitoring System,” filed Feb. 15, 2013, the entire disclosures of each of which are hereby expressly incorporated by reference herein.

### FIELD OF DISCLOSURE

The present disclosure relates generally to a personal health monitoring system and more particularly to the use of a device or system to monitor a person's physiological condition or attributes and use that information to diagnose, predict and/or advise a user on his or her well-being.

### DESCRIPTION OF RELATED ART

Systems for monitoring various physiological conditions for an individual are fairly common. One such system available today includes a wearable health monitoring system that includes sensors that are integrated with a telemedicine system. For this system, various sensors are attached to an individual and the sensed data that is created is communicated to a phone. Once collected at the phone, the data is then sent to a remote server where doctors and trained physicians can analyze the data. Similar types of systems have also been used by athletes for measuring their physical attributes during training. Again, these systems collect the sensed data and then send the information to a tablet or computer to analyze the data. Sometimes a phone is used to get the data to the tablet or computer. However, in these type of systems, performance is measured, not the health and well-being of the user.

What is needed is a health monitoring system that is integrated into a cellular phone or a tablet having cellular or internet communications which allows a user to collect a wide variety of data, including various physiological conditions, and to analyze the data for the purpose of determining the well-being of that user. Thereafter, should the need arise, the collected data, analysis, or other information could be sent to a treating physician (using the cellular communication feature) for further evaluation. This type of system would not only be convenient and practical, because everyone is currently using their cell phone or tablets for a variety of other applications, but would also be beneficial because it would allow the user to maintain control and security over the personal individual data. As a result, a great deal of expense in time and money could be saved by avoiding unnecessary doctor visits.

### SUMMARY

This invention relates to a personal health monitor device which may be a cellular phone or tablet with internet connection. The device includes a memory for collecting and storing values of various physical and environmental attributes collected from or about an individual. The device further includes a processor for quantizing each attribute in such a way as to indicate a baseline and a normal range for that attribute. Once the baseline and normal range has been identified, deviations from the baseline and/or normal range are identified. These deviations are then used to indicate possible symptoms indicating the well-being of the individual. These symptoms are compared to symptoms of known illnesses to determine if the individual may have a known illness. The results of these comparisons may be displayed to the individual on a display. Should the individual wish to send the results to trained medical personal, he or she may transmit the results or any attributes which led to those results using internet or cellular communications. In some cases, the personal health monitoring system may include one or more expert engines and/or health predictive modules that may use the personal data, including the personal attribute data, environmental data, baseline and normal range data, deviation data, etc. to perform health diagnostics (e.g., to identify current health conditions of the user) or to predict future health issues (e.g., to predict the onset of an epileptic attack). The expert engine and/or predictive modules may use one or more models that are generated using the personal and environmental data, wherein the models determine or reflect various cycles detected in the person's body, e.g., blood cycles, oxygen cycles, food cycles, urination cycles, breathing cycles, etc., and/or reflect detected highly correlated relationships between various of the physical and/or environmental parameters and health issues or health conditions. These models can be periodically created and modified based on newly collected data to reflect the current operation or state of the user's body and can be used to perform health diagnostic and predictive analyses.

### BRIEF DESCRIPTION OF THE DRAWINGS

FIG. 1 is a diagram of a personal health monitoring system capable of collecting and storing data representing a user's personal attributes from different sources for the purpose of analyzing the attributes using a processor to determine and display the well-being results of the user.

FIG. 2 illustrates a display of a phone or tablet showing functions that may be selected for monitoring the health of a user.

FIG. 3 shows an example of a series of health assessment questions that the monitoring device could query the user for to collect additional information regarding the user's weight and blood pressure and some calculated results such as body mass index and general assessment of blood pressure based on those answers.

FIG. 4 shows an example of a series of questions used to query the user regarding medical history of the user.

FIG. 5 illustrates another display of a phone or tablet showing functions that may be selected for monitoring the health of a user.

FIG. 6 shows a graph illustrating the measurements of an attribute such as blood pressure for the user over a 24 hours period.

FIG. 7A shows a graph illustrating the measurements of the baseline and trend of an attribute such as blood pressure of the user over a period of 30 days.

FIG. 7B shows a graph illustrating the measurements of the baseline and trend of an attribute such as blood pressure of the user over a period of 5 years showing the effectiveness of medication taken by the user to lower the user's blood pressure.

FIG. 8 illustrates another display of a phone or tablet showing suggestions generated by the health monitoring system that the user may want to consider as a result of the historical analysis of the user's blood pressure.

FIGS. 9A and 9B depict a flowchart illustrating the process of analyzing the measured attributes of the user to determine the well-being of the user.

FIG. 10 illustrates a block diagram of a further personal health monitoring system having an expert engine that is used to perform diagnostics and a predictive module to predict future health issues using personal health models.

FIG. 11 illustrates a cloud based personal health system that operates using data from multiple people.

FIG. 12 illustrates a flow chart of a routine that performs predictive analysis of personal health issues using data/statistics from multiple people that may be implemented in the system of FIG. 11.

### DETAILED DESCRIPTION

An individual health monitoring system operates to collect and process data related to the health of an individual and operates to perform personal health diagnostics or condition analysis and to predict future personal health issues. The individual health monitoring system is preferably integrated into a cellular phone for personal use, but may be implemented in any other type of computing/storage device, such as a laptop, a standalone personal monitoring device, a cloud based system, etc. Thus, while a cellular phone is shown and described as the preferred embodiment, a tablet or similar computing device that has cellular communications capabilities or internet access is contemplated to be used. Generally speaking, the phone or tablet includes or has access to a large database for storing some or all of the data collected from any number of personal sensing devices or personal input devices that are used to collect data related to physical attributes of the individual and includes software applications that can be called upon by the user (or automatically) to evaluate the collected data to determine the well-being of the individual. Results of the analysis may include diagnoses of current personal health issues, predictive health analysis that predicts possible future health issues, conditions or concerns, providing suggestions or recommendations for improving that individual's health, etc., any and all of which can be displayed on a display screen of the phone or tablet, provided via a voice generation unit on the phone or tablet, sent to the user via a text message or an e-mail message, etc. Depending on the results of the analysis, the user may select to send the results and/or data used to generate those results to a treating physician using standard cellular communication or any internet access features. In many cases, the database may also store other information useful in performing personal heath diagnostics or predictions, including for example, informational data regarding prescribed (or other) drugs taken by the user and their known side effects, generally known diagnostic data, such as disease or sickness symptoms, previously detected diagnostic conditions, etc., and this data may be accessed or retrieved using the cellular or internet connection and used to diagnose personal health issues or to predict future personal health conditions. As an example of a preferred feature of the system, the physical attributes of the individual that are monitored may be used to determine if any undesirable side effects exist as a result of taking medications, and, if so, the system may generate an alert that is provided to the user.

Preferably the personal health data is collected wirelessly from the sensing devices, but could be downloaded directly from the sensing devices using typical wired connections such as those that use USB, Firewire, or any other communication protocol. The personal health monitoring system could be used in a continuous mode, for collecting individual data that is collected or uploaded continuously, or could be operated in a periodic mode to periodically collect data from a sensing device, which is capable of collecting and potentially storing the information. Information may also be manually entered in by the user such as using a keyboard, a mouse and input screen, a voice input system that uses voice recognition software, etc. In one example, typical questions often provided by doctors and required to be filled out by a user could be used as a means for collecting personal information.

The database used for storing this information should be large enough for storing large amounts of data regarding the physical attributes of the individual and should include at least one, but preferably may include data related to or indicative of many such physical attributes, such as body temperature, blood pressure, humidity, ECG, breathing, blood sugar, heartbeat, administered medications, past medications, etc. Preferably the data is stored in association with the time that the data was created and/or collected to provide a timeline for the data. This feature allows for historical timelines of data to be evaluated as well as to identify and notify the individual when fresh data is needed to properly analyze the well-being of the individual. Using this data, normal baseline and normal range data that is unique for that person can be identified. The normal baseline for a particular attribute could be calculated as the average measurement for the day, the average measurement over a period of 30 days or any other medically acceptable range for a given attribute. An acceptable deviation or range from that base line could vary depending on what would be considered normal for that particular attribute. For example, not everyone's average, normal or median body temperature is the same. As appreciated by those skilled in the art, what may be a normal baseline or range for one person is not necessarily the normal baseline or range for another. The same consideration would apply to blood pressure, heart rate, breathing, blood sugar, etc. Still further, the system can detect or determine trends for the baseline and ranges and can perform analysis on or using these trends. For example, depending on the time of day, age, etc., blood pressure tends to trend in different directions. In the morning, for example, blood pressure is usually at the highest. Moreover, as a person ages, blood pressure trends upwards, especially if the person has a family history of high blood pressure. Understanding these normal ranges and trends can be very important for diagnosing a person's well-being, as well as for understanding how to properly prescribe medication if needed. Plus, by monitoring the baseline, ranges, and the trends, the user (and/or a user's doctor) can monitor the effectiveness of the type and/or dosage of the medication being taken or prescribed. Further, as would be appreciated by those skilled in the art, other applications are possible. For example, the health monitoring system could be used for those that work in toxic environments and, in this case, health effects related to exposure to those toxins could be monitored.

It is also preferred that additional information could be stored to further aid in the analysis of a person's well-being by including information such as a user's medical history including, if possible, family medical history and personal medical history. Additional information that is unique to the individual could also be entered by the individual to form a more complete data set. For example, information on food consumption, types of food, exercise information, sleep information, weight, prescription/medication (current and past), bodily fluid discharge, etc. This information could be formatted in such a way as to allow it to be easily accessed and read as necessary by an application analyzing the data, as well as to be fully searchable. For example, being able to search and review current and past prescriptions can be critically important to determine the compatibility of new medication.

One of the benefits of such a system is that using the artificial intelligence of the system, the system could discover, query the individual, or automatically identify symptoms, rather than asking the individual to recognize the systems for himself when a well-being application is selected. Oftentimes an individual does not understand or appreciate what symptoms he or she should identify as being important. Another advantage is that the system could effectively be operated as a personal doctor's aid, by providing medical alerts or early detection of diseases or harmful conditions that could even include reactions to current or new medications. The personal health monitoring system could also detect when the user missed taking prescribed medications and alert the user. In general, the system would enable an individual to monitor his or her own health and only consult a doctor if the need exists. Many unnecessary doctor visits could thus be eliminated. Further, by centralizing all of the data on a personal device, such as a cellular phone or tablet, the information can be kept confidential, secure, and under the control of the individual. If that individual wishes to share that information, the cellular or internet communications feature provides a convenient way to share information or data with a treating physician. For example, if a patient that has high blood pressure has a prescription that is about to run out, the person may monitor his own blood pressure with a device approved by his physician and then send the collected data to the physician, who can then approve the proper dosage and renew or change the prescription at the pharmacy directly, rather than making an appointment to get a refill. Both the physician and the patient save a considerable amount of time, which results in cost savings to the patient. This scenario is just one example of the utility and benefit of the personal health monitoring device/system described herein. Of course, one skilled in the art would appreciate or could envision many other such examples of savings of time and money in the health care industry that could be employed using such a device/system.

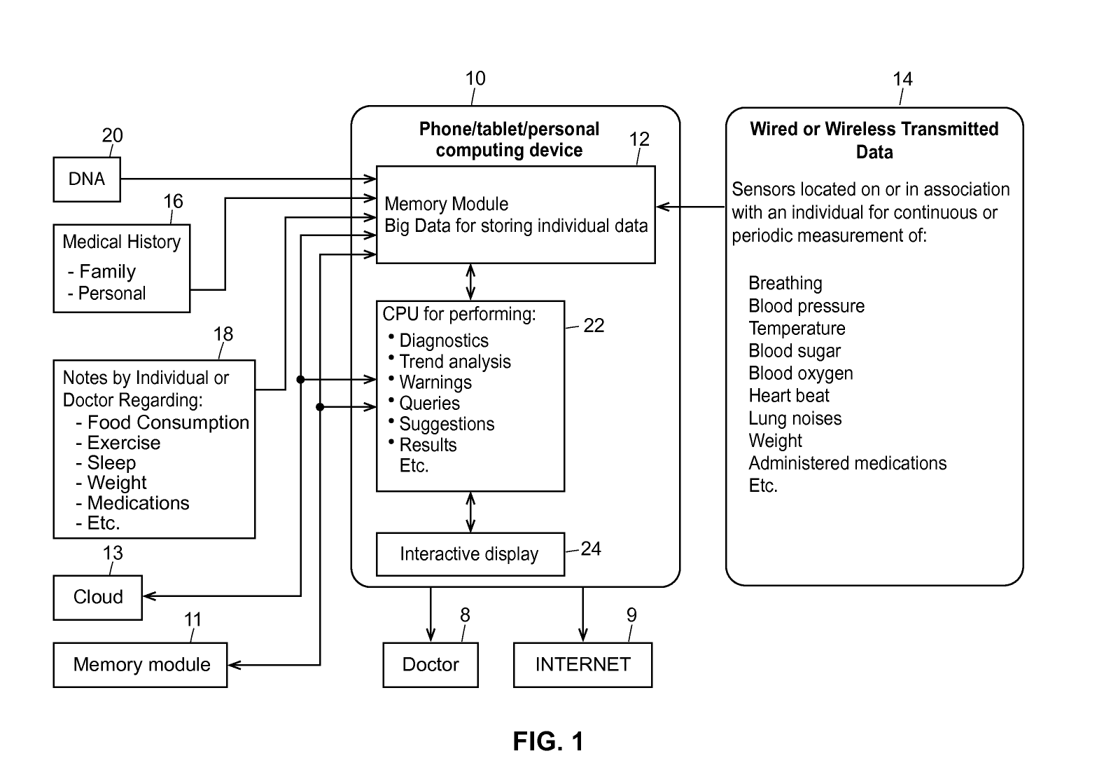

Referring now to the FIG. 1, a schematic diagram of a phone 10 is shown with data being communicated to it from various sources. As mentioned above, it is contemplated that a tablet or similar computing and display device, or a stand-alone, wearable device configured primarily to implement the personal health monitoring system described herein could be used instead of a phone. It is preferred that such a device have a cellular communications feature to allow sharing the collected information with a treating physician 8 or to include an internet or other wireless (or wired) communication system to enable access to or to retrieve data using the internet connection 9. As shown, the phone 10 includes a high capacity memory module 12 for storing large amounts of individual data. Preferably the data is time stamped or references a time when the data was actually collected. While FIG. 1 shows an internal memory module 12 for storing the individual data, one skilled in the art would appreciate that this data could be stored using an insertable flash card, a Sims card, or a similarly high capacity memory 11 connected to the phone or tablet 10. Further, the data could be remotely stored using what is generally referred to as cloud technology 13, which is commonly used to store data using conventional cellular or internet technology and which may then be called upon by the software application selected by the user to evaluate the collected data to determine the well-being of the individual. Alternatively, the artificial intelligence for evaluating the data collected could be stored and implemented within the cloud 13, where this software can periodically process the data and send the results of such evaluation to the user's phone or tablet 10. To facilitate storage of large amounts of data, many different known types of data compression techniques could also be used and such data compression techniques are well known by those skilled in the art. For example, loss-less compression and lossy compression techniques can be used and have long been generally used to avoid storing unnecessary information and thereby increase the capacity of the memory available. Similarly, there are other known techniques to help in extrapolating data when data is missing in the timeline and these data extrapolation or interpolation techniques may be used as an aid to analyze the current data for the well-being of the individual as would be appreciated by one skilled in the art.

Data is collected from at least one sensor and preferably several sensors 14 used to collect data in association with the physiological condition or attributes of the individual. The more types of information over time that are collected, the better the opportunity to data mine the collected data for creating baselines, ranges, trends, diagnostics, prognostics, etc. For example, breathing, blood pressure, temperature, cholesterol, blood sugar, blood oxygen, heart beats (and/or heart rate), lung noises, weight, administered medications, etc. are some of the parameters contemplated. Many others are possible as would be appreciated by one skilled in the art. The sensors 14 can be located on or in association with the individual user by way of one or more of a vest, an armband, a wrist band, an ankle band, etc. Many of these devices are readily available. For example, Best Buy currently advertises and sells a host of wireless devices that sense and monitor individual physiological conditions. One such device is a wireless activity and sleep tracker. Another device is a “BodyMedia—Fit Link Armband” that measures calories burned, body temperature, steps and sweat, sleep quality, etc. and is wireless in nature. Still others sell monitors for measuring blood pressure and blood sugar. The sensors 14 could also be intrusive to the individual such as pace makers or other devices which may, for example, deliver medication to the individual. In a preferred embodiment, the data is collected by the sensing device(s) 14 and is transmitted wirelessly to the data storage module (11, 12, and/or 13). However, data may be communicated from the sensing devices 14 to the data module 12 in the phone or tablet 10 via wires, such as a USB cable. In each case, a time associated with when the data is collected is stored.

Because each individual is unique, collecting relevant data from other sources is also preferred. As illustrated in FIG. 1, data including medical history for both family and personal 16 is also preferred. Additional information about the individual may also be provided. For example, information regarding the individual's food consumption, exercise routine, sleep habits, weight, medications, etc. 18 may also be provided. DNA information 20 may also be important information in the future for properly analyzing the data and could be included. Taking samples of bacteria from different areas of the body for analysis is also contemplated. As well-being programs are developed, one skilled in the art would realize that other data may be needed and collected. In other words, as the artificial intelligence for analyzing the data improves to detect the well-being of a person, additional data or data collected in a new way may be necessary to complete the analyzes, and is contemplated for use by the system described herein.

In a preferred embodiment, the processor 22 of the phone or tablet 10 is used to initiate applications which access the data from the memory module (11 and/or 12) or the cloud 13 and perform calculations using the collected data to assess the health or well-being of the user depending on the health or well-being programs selected or continuously operating in the background monitoring the user's health. However, it is possible for a second processor to be provided and dedicated to these applications. As an example of the type of applications that could be provided, one could include a general diagnostics application on the well-being of the individual's blood pressure to determine if there may be issues of high blood pressure indicative of heart attacks, strokes, heart failure, kidney disease, stress, etc. Similarly, an application could be provided to assess the individual's blood sugar to identify issues with diabetes. Because blood sugar can fluctuate throughout the day, understanding a person's sugar levels over time will be important in many diagnostics. Trends regarding the above conditions as well as trends regarding various other health conditions such as good and bad cholesterol could also be determined and analyzed. General health assessments, warnings, suggestions, and recommendations could be provided and are a few of the benefits of these applications. It should be understood by one skilled in the art that, while the examples of blood pressure and sugar levels are being monitored, all of the data from a variety of different data sets (blood pressure, temperature, heart rate, etc.) could and should be used in the analysis and diagnostics of the individual. In other words, analysis of the individual is not limited to looking at just the data set for a given personal attribute. Numerous other medical applications to assess the individual's well-being are also possible but not mentioned here, and one skilled the art may develop many such applications which could be provided to and downloaded by the phone or tablet 10 and used in any of the manners described above. The resulting analysis and interaction with these applications can be shown on an interactive display 24 that is commonly available on the phone or tablet 10 and may be shown in many useful and creative ways. Graphs or tables showing normal ranges, baselines, trends, or statistics showing data are possible. Colorful alerts, warnings or suggestions may also be displayed. Sound alarms are also possible. Further, forms can be created and displayed for querying the individual for more information to complete the data set for analysis. It should become clear that the personal health monitoring system could store and allow the user to access many different expert medical applications to thereby leverage the wealth of medical knowledge now available in evaluating and diagnosing the data to determine the well-being of the individual.

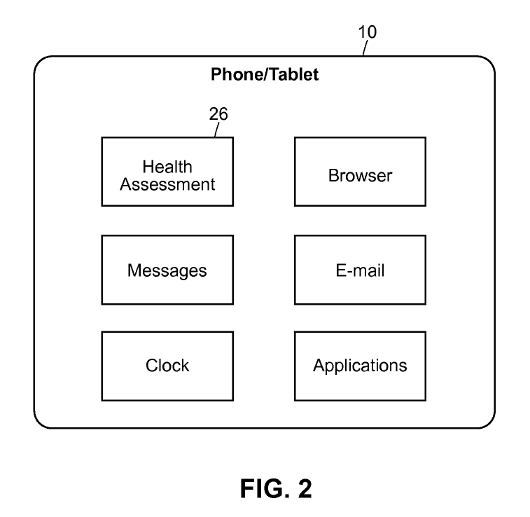

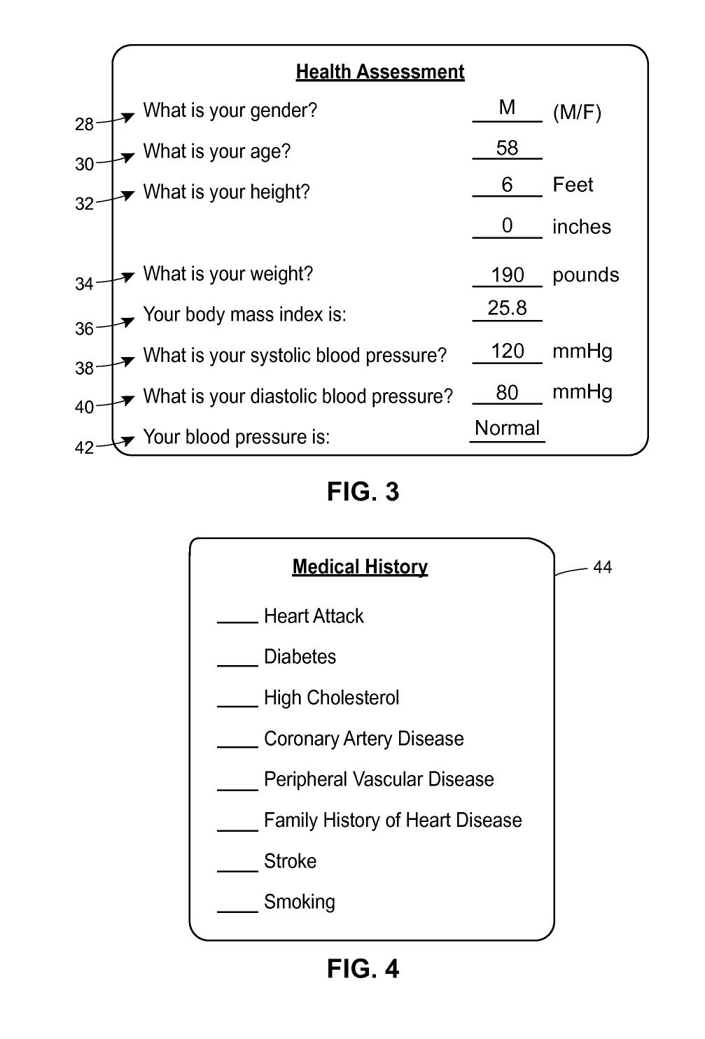

Referring to FIGS. 2-7, some example screen shots of a cellular phone or tablet 10 are shown and are used to illustrate how a user might: select one of several potential health assessment applications that could be downloaded and stored on his phone; be queried to enter information; and see displays showing the results of the analysis of the assessment. One skilled in the art would understand that many other types of applications could be stored and selected by a user, questions could be asked of the user to help determine the well-being of the user, and displays showing different parameters, trends, etc. could be shown. As one example of an application that could be stored and selected, FIG. 2 shows a general health assessment program 26 using the interactive screen of his or her phone or tablet. Selecting this application could result in a query for information from the user. As shown and illustrated in FIG. 3, a query could ask for or include the user's gender 28, age 30, height 32, and weight 34. This information could then be used to calculate the user's body mass index 36. Additional information could be queried such as blood pressure, including the systolic and diastolic values 38 and 40. Based on this information, a health indication 42 can be automatically displayed as high, normal or low blood pressure to indicate the well-being of an individual. While this example demonstrates how a user would be queried to enter information regarding the user's blood pressure measured results, this information could be transmitted to the phone wirelessly or by wire from one or more measuring devices as suggested above. As already mentioned and as will be appreciated by one skilled in the art, a tablet or like device could be used in place of the phone for performing the general health assessment.

Referring now to FIG. 4, queries could also be made for medical history. As an example, a form 44 is shown in FIG. 4 asking the user to provide information on his medical history by interactively placing a check next to the appropriate items shown. One skilled in the art would appreciate that there is an enormous amount of medical history information that could be obtained from the user to help in the medical assessment of the well-being of the user. These forms could be arranged and could appear in an endless variety of ways. The present form shows spaces that can be checked, that would allow the user to select past medical issues or current conditions that would apply to that individual, such as previous heart attacks, diabetes, high cholesterol, coronary artery disease, peripheral vascular disease, family history of heart disease, stroke, and smoking just to name a few that are possible.

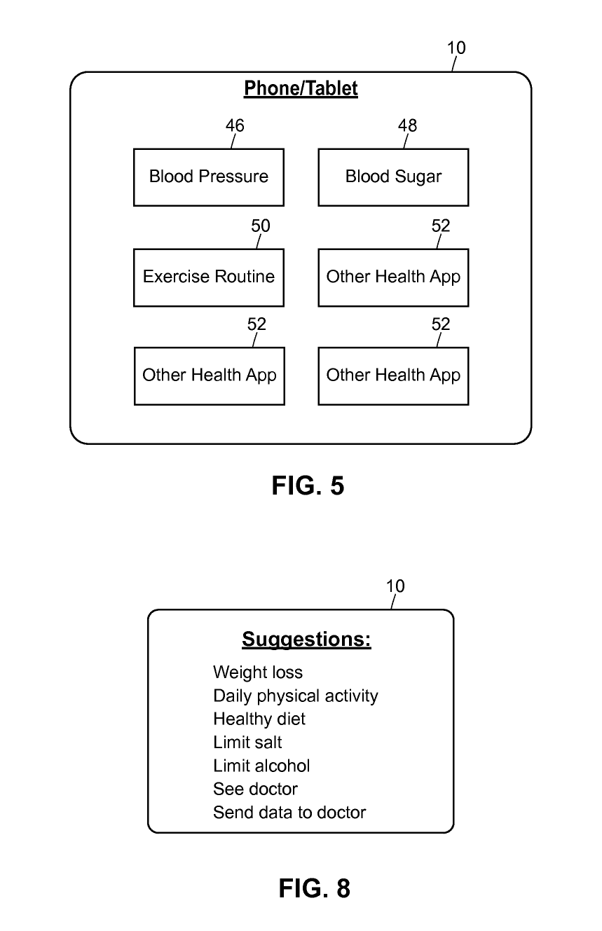

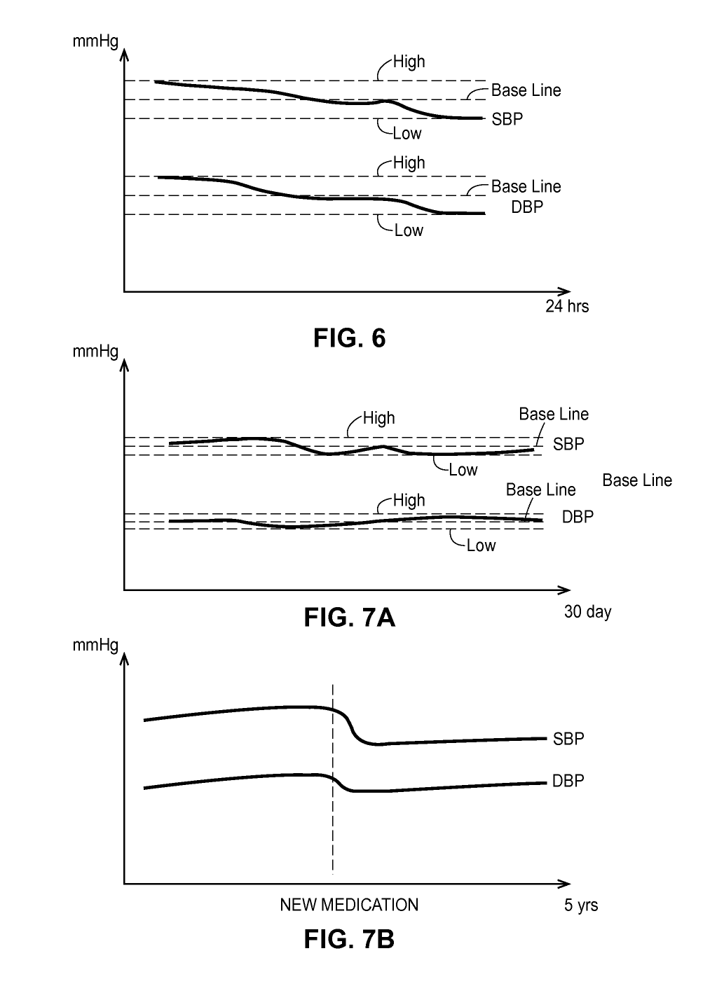

Turning to FIG. 5, the user could select applications from a main menu shown on the phone or tablet 10 (shown in FIG. 2) to display a variety of health monitor applications such as blood pressure 46, blood sugar 48, exercise routine 50, and others 52, as shown in FIG. 5. As one skilled in the art would appreciate, there are a variety of health monitoring applications that could be created, stored and selected by the user and the applications shown in FIG. 5 are merely examples of the types of health monitoring applications that could be installed and selected by a user. Many others are possible and contemplated. As an illustration, the user could select the blood pressure application 46. Based on the historical information on measured blood pressure, a graph could be displayed showing the user his or her blood pressure over the last 24 hours as illustrated in FIG. 6, over the last 30 days as illustrated in FIG. 7A, or over any other desired range. FIG. 7B is provided to illustrate the results and effectiveness of a user that has taken medication to reduce his blood pressure. In the alternative, it should be appreciated by one skilled in the art that the health device/system could be used to measure the effects of a user exposed to chemicals in a toxic environment. The attributes of the user can similarly be monitored.

Rather than depicting a graph as shown, tables or other manners of showing this information are possible and would be appreciated by one skilled in the art. Depending on the results of the blood pressure data, the personal health monitoring system could provide suggestions to the user for improving the results of the user's blood pressure, as illustrated in FIG. 8. These suggestions could include such things as a recommendation that the user lose some weight, increase his or her daily physical activity, improve the user's diet (with recommendations of the types of foods that the user should avoid or include in their diet), limit his or her salt or alcohol intake or see a doctor regarding his or her blood pressure or medication therefor. A variety of other suggestions or recommendations are possible and would be realized by one skilled in the art depending on the data collected and analyzed. Further, the health monitoring application could further refine these recommendation or suggestions by using the historical data collected from other measured physiological attributes of the individual. For example, the application could eliminate some of these suggestions or recommendations to one or two for the user to consider and follow. Therein lays one of the benefits of this concept. By collecting data uniquely from one individual, the recommendations or suggestions can be tailored, based on that data, to the needs of that individual. For example, it may be that the user has a healthy diet and is in great physical shape and that the only recommendation may be that they consult their doctor. If the only recommendation or suggestion is to see a physician, then the information that resulted in this conclusion could be sent to the physician using e-mail or internet features common to most cellular phones or tablets. In this manner, the physician may review the data prior to the visit, may decide that a visit is not necessary or should be delayed, or alternatively that there is a problem and that a visit should be scheduled immediately, or that some test should be performed before the visit, etc.

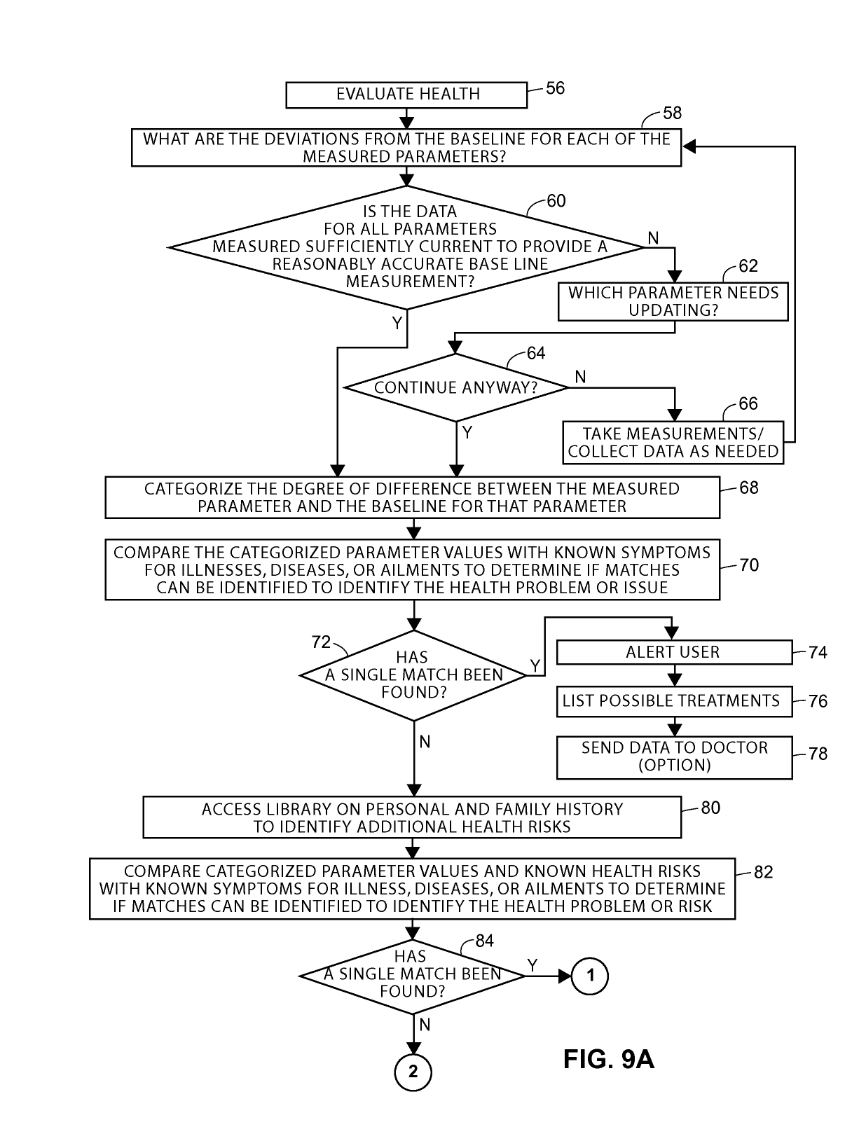

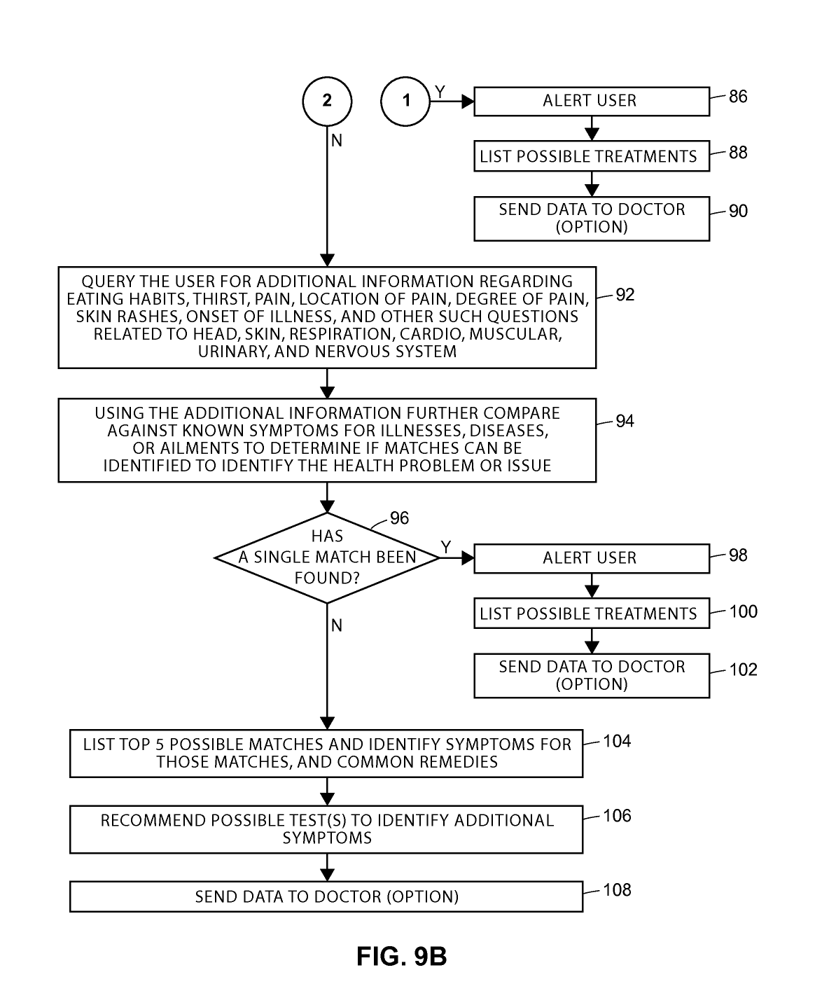

Referring now to FIGS. 9A and 9B, the following illustrates how such a system might analyze the information to illustrate how the health monitoring system can narrow down the possible causes of an ailment or diagnose the ailment. To start, the user can select the health assessment button 56 to start the analysis. The first step in the process is to look at the measured parameters and identify deviations from the baseline or range for each of the measured parameters 58. A determination is made to see if the data is current and sufficient to provide a reasonably accurate baseline and range measurement. For this example, it is assumed that there is a sufficient collection of data over a period of time to determine a reliable normal basis and range for each parameter measured. It is also assumed that there is sufficient data to provide a trend for these parameters too. The actual amount of historical data needed to provide a reliable trend, baseline, and range measurement may depend on the parameter being measured or considered. Alternatively, medically acceptable baselines and ranges could be used. The system preferably has a default which would recognize when the measured information is sufficiently current enough to be used in any evaluation 60. If not, then the attribute that needs updating is identified 62 and a decision is made by the user to either continue or to collect the necessary data before proceeding 64, 66. For example, if blood pressure has not been taken for a period of a couple of months, then a recommendation could be made to the user to take several blood pressure readings over the next couple of days to provide more current data for the evaluation. If one of the parameters is cholesterol, then data collected every couple of months may be more than enough to provide a reasonable amount of data for determining a baseline measurement. In a preferred embodiment, the system would recognize when new data is needed to update the system with reliable data for evaluating health before the user even requests a health evaluation and sends the user a message or puts the user on notice that new data is needed. The system could account for this choice by providing a weighted value for these parameters when they are not current.

As shown, the user has a choice to proceed with the analysis with slightly outdated data or no data at all. Medically acceptable ranges such as 120 over 80 for blood pressure could be used to complete the analysis when there is no data, the data is incomplete or when the data is outdated. For the cases in which the user decides to continue with the analysis, it is preferred that, at the end of the analysis, recommendations are provided to the user to collect more data regarding certain parameters for a more accurate analysis.

Deviations from the baseline and/or the normal range can be classified or categorized by identifying the deviation as normal, a little high, high, a little low, low or by scaling the deviations placing a scaling value such as 1 to 10 between normal and high and similarly −1 to −10 between normal and low 68. Other manners of scaling, classifying, or categorizing the deviations from normal are possible. For the present example, it is assumed that the currently measured parameters indicate that blood pressure is a low, temperature is normal, heart rate is a little high, respiratory is normal, weight is a little low, oxygen saturation level is normal, and glucose is high. At this point, the system could access a library of known illnesses, diseases, or aliments to compare their known symptoms to the identified categorize parameters to identify possible matches for candidates that may be causing the user to have poor health 70. If the list of possibilities is significant, more investigation may be necessary 72. Typically with a limited number of parameters measured, more information will be need. If, however, a match is found, the user can be alerted 74 and a list of possible treatments could be provided 76. Additionally or alternatively, this information including the data and the results can be sent to a doctor 78. If no match is found at 72, more information is needed to complete the analysis.

Next the library of information on personal and family history is accessed (at the block 80). Such things as medications that the user is taking and their possible side effects are considered to determine if such side effects would result in some or all of the conditions indicated by the measured parameters 82. Similarly other historical conditions are considered such as race, gender, age, past medical history, prior illnesses, previous surgeries, alcohol usage, smoking habits, exercise activity, dietary, allergies, etc. For the present example, it is assumed that the user has a history of being overweight and his or her glucose trend over the past year has been running on the high side. Based on the previous analyses, these factors appear to be significant factors when combined with the measured parameters and then compared with known symptoms of known illnesses, diseases, or aliments 84. If a match is identified, the user is alerted 86 and a list of possible treatments could be provided 88. Again, the user has the option to send some or all of the information to his doctor 90. If no match is found, more information will be needed to continue with the analysis 84.

To help narrow down the illness, a list of questions is preferably asked of the user 92. These questions may be the typical questions that are asked at a doctor's office on a first visit for an illness but could include other questions. These questions can include such things as: Is there any pain? Where is the location of the pain? What is the degree of pain on a scale of 1 to 10? Are there any skin rashes? Did the illness onset come quickly or slowly? Is there congestion? Is there a cough, head ache, tired, restless, etc.? Generally, the typical questions are directed to the head, skin, respiration, cardio, muscular, urinary, and nervous system. For the present example the user has noticed an increase in the need to urinate.

Based on this line of questions, along with the current parameters measurements, the personal and family history and the questions, a preliminary diagnosis might be determined and recommendations made or further questions may be asked 94. For example, questions regarding whether the user has been eating normal, has excessive hunger, excessive thirst, pain, etc. Once these questions have been answered by the user, a determination is made as to whether there is a match of symptoms to a known illness 96. If not, the system may perform further queries 92, 94. For the present case it is assumed that there was excessive hunger and thirst. These symptoms, when combined with the above data, help narrow the analysis and would suggest that the health issue may be related to diabetes, urinary tract infection, or other disorders that may require the attention of a doctor. The user is alerted 98 and possible treatments are identified 100. The analysis and the basis of this diagnosis could be downloaded and then sent to the user's doctor using the e-mail or internet features of the phone or tablet 102. If the diagnosis were to be something less threating, such as a cold or flu, common remedies or over the counter medications might be suggested. In all cases, it is a preferred embodiment that the health system identifies the possible causes for health problems, the symptoms of those causes, and/or the list of possible treatments. In the case where no match has been found, several of the closest matches, for example the top five matches along with their symptoms and common remedies could be brought to the attention of the user 104. Further, recommendations on the type of tests that could help identify the illness could be displayed to the user 106. All of these results can then be sent to the doctor 108.

The above example is only illustrative and the personal monitor health system described herein is not limited to finding or diagnosing illnesses, but also may look for side effects of prescription and non-prescription medications. The system could also look for conflicts or the effects of combining medications and alert the user. In these cases, it is preferred that the data module include a library that contains at least a list of known side effects of medications that the user is taking so that it can be compared to the measured parameters to look for these side effects and to ask questions of the user for more information should some of these side effects be detected. For example, questions similar to those above or directed specifically to the indicated side effects of the medication could be asked of the user. If the issue relates to a possible reaction to a current medication that the user is taking, an alert is given to the user along with the known side effect of that medication. The user can thereafter send this information to his or her doctor using e-mail or internet capabilities of the system.

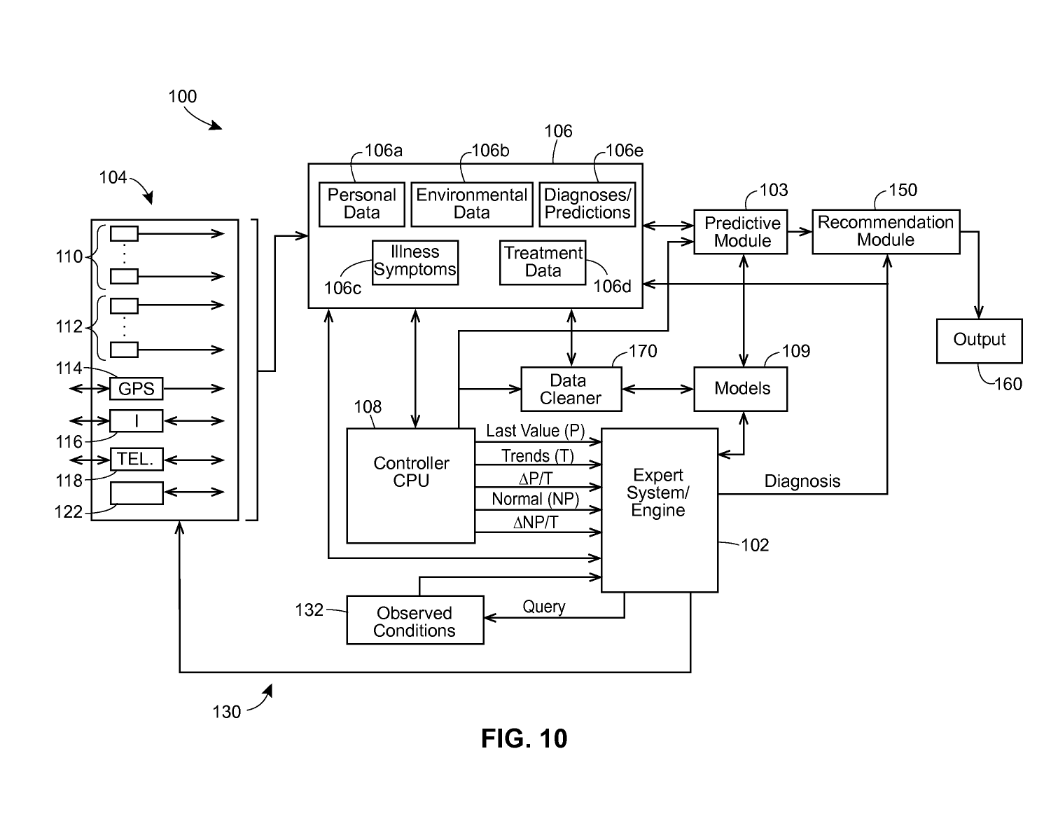

FIG. 10 illustrates a block diagram of a further example of a personal health monitoring system 100 having an expert engine 102 that is used to perform diagnostics and a prediction module 103 that is used to predict potential future health issues or conditions in a more comprehensive manner. In particular, the personal health monitoring system 100 includes an input unit 104, a database 106, and a controller/CPU 108, in addition to the one or more expert engines 102 and one or more prediction modules 103. The input unit 104 may include or is attached to various sensors 110, that measure body related or physical parameters and may include, for example, body temperature sensors, pulse rate or heartbeat sensors, step monitors, blood sugar (glucose) level sensors, carbon dioxide sensors, breathing sensors, or any other type of sensors that detects or measures a physical parameter of any part of the user's body, including any of those mentioned above with respect to the system of FIG. 1. The input unit 104 may also include ambient environment sensors 112, such as pollen sensors, temperature sensors, humidity sensors, smog sensors, radiation sensors, etc. The environmental sensors 112 may measure or detect any environmental parameter associated with the environment in which the user is present. Additionally, the input unit 104 may include a global positioning system (GPS) unit 114 or other position detection unit, to detect the location of the user at any particular time using, for example, global positioning signals, cell phone tower positioning signals, etc. Additionally, the input unit 104 may include or be connected to other sources of data input, such as an internet connection 116, a wireless phone connection 118, etc., and may have access through these communication connections to other types of data stored elsewhere, such as the temperature, pollen count, humidity, rainfall, smog, etc. of a particular location, symptoms associated with medical conditions or illnesses, known drug interactions, etc. These types of inputs may be associated with or used in conjunction with the GPS sensor 114 to determine or ascertain environmental parameters associated with the location of the user at any particular time, such as the ambient temperature, pollen count, smog level, etc. of the location of the user at any particular time. These inputs are especially useful when particular types of environmental sensors are not available as part of the sensors 112. Still further, the input unit 104 may include user input/output devices 122, such as a display screen, a microphone/speaker, a voice input unit (including a voice recognition unit that converts voice or dictation to data to be stored for the user), a keyboard, a mouse, a trackball or other user operated data input mechanisms. These devices may be used to enable a user to input data in response to questions, forms, prompts (e.g., voice or display screen prompts) displayed or otherwise provided by the device 100.

Likewise, as illustrated in FIG. 10, the database 106 stores various types of data that is collected by and/or determined by the system 100 or that is otherwise used by the system 100 to perform personal health monitoring in the form of health diagnosis and health predictions. As indicated, the database 106 may store any and all of the data collected by the input system 104, as well as data generated by other components the system 100 itself, in the form of diagnosis, trends, baselines, deviations from baselines, etc. As illustrated in FIG. 10, the database 106 may store personal physical data 106A that is measured by or collected by the system 100 via the input system 104, e.g., blood pressure; oxygen saturation; cholesterol; weight; body temperature; glucose levels; drug, food and/or liquid intake data; exercise data; sleep data; age; sex; medical history; allergies; drug, food and other reactions; family medical history data; etc. Additionally, the database 106 may store ambient or environmental data, such as ambient temperature, humidity, pollen count, GPS data, etc. for the user which data may be obtained via any of the sensors 112, the GPS unit 114, via the internet or phone connections 116, 118, etc. Still further, the database 106 may store diagnosis data 106C, including illness and disease symptoms, effects, markers, etc. for any number of possible illnesses or diseases. For example, symptoms of mononucleosis may include fatigue, general feeling of unwellness (malaise), sore throat, or strep throat that does not respond to antibiotic use, fever, swollen lymph nodes in the neck and armpits, swollen tonsils, headaches, skin rash, soft swollen spleen. Moreover, for this illness, the database 106C may store that the virus has an incubation period of approximately four to six weeks, although in young children this period may be shorter and that signs and symptoms such as fever and sore throat usually lessen within a couple of weeks, although fatigue, enlarged lymph nodes and a swollen spleen may last for a few weeks longer.

In a similar manner, the database 106C may store the symptoms of viral pneumonia as low fever, chills, muscle aches, fatigue, enlarged lymph nodes in the neck, chest pain, sore throat, and coughing that usually brings up only a small amount of mucus. The database 106C may store bacterial pneumonia symptoms as high fever, cough with thick greenish or rust-colored mucus, shortness of breath, rapid breathing, sharp chest pain that gets worse with deep breaths, abdominal pain, severe fatigue, chills, heavy sweating, and mental confusion.

As another example, the database 106C may store symptoms or conditions related to lung disease/respiratory problems and asbestos exposure. Generally speaking, asbestos is a group of minerals with thin microscopic fibers. Because these fibers are resistant to heat, fire, and chemicals and do not conduct electricity, asbestos has been mined and used widely in the construction, automotive, and other industries. If products containing asbestos are disturbed, the tiny fibers are released into the air an when the asbestos are breathed in, they can become trapped in the lungs and stay there for many years. Over time these fibers can accumulate and lead to serious health issues.

Additionally, the database 106C may store lung cancer symptoms, such as coughing (e.g., a persistent cough that does not go away or changes to a chronic “smoker's cough,” such as more coughing or pain, coughing up blood, coughing up blood or rust-colored sputum (spit or phlegm); breathing difficulties including shortness of breath, wheezing or noisy breathing (called stridor); loss of appetite which may lead to unintended weight loss; fatigue (e.g., feeling weak or excessively tired); recurring infections like bronchitis or pneumonia; and flu symptoms, such as high fever, headache, tiredness/weakness, dry cough, sore throat, runny nose, body or muscle aches, diarrhea and vomiting (more common for children).

Still further the database 106C may store signs or symptoms of campylobacter infection (i.e., food poisoning). Generally, campylobacter is a bacterium that causes acute diarrhea. Transmission usually occurs through ingestion of contaminated food, water, or unpasteurized milk, or through contact with infected infants, pets, or wild animals. The database 106C may store symptoms of campylobacter as including diarrhea (sometimes bloody); nausea and vomiting; abdominal pain and/or cramping; malaise (general uneasiness) and fever.

The database 106C may store symptoms of kidney stones, including waves of sharp pain in the back and side or lower abdomen that may move toward the groin or testicles; an inability to find a comfortable position; pacing the floor; nausea and vomiting with ongoing flank pain; blood in the urine; and frequent urge to urinate. Also, sometimes an infection is present, and may cause the additional symptoms of fever and chills, painful urination and cloudy or foul-smelling urine.

Of course, these are but a small number of sets of symptoms of various illnesses and diseases and symptoms for any other number of diseases, illnesses and conditions can be stored as well or instead. Moreover, as will be understood, indications of many of these symptoms cannot be measured directly and so have to be entered by the user manually or via a voice input mechanism, via an ask and answer screen, or a pop-up window that may allow the user to check off the symptoms that are currently observed. Moreover, the expert system 102 or the predictive module 103 may inquire of the current or past observed conditions when performing a diagnosis or prediction.

The database 106 may also store treatment data 106D including procedures, remedies and other treatments for diseases, illnesses, or other medical conditions, including for example, the names, dosages, side effects, etc. of drugs that are known to be used for the treatment of illnesses, diseases, and other personal medical conditions (e.g., muscle, head, stomach, bone, etc. aches and pains). The database 106D can also store drug and food interactions. Still further, the database 106 may store data pertaining to diagnoses and predictions 106E for the user made by the personal health monitoring system 100 itself and any data generated as part of that process. For example, the database 106E may store previously determined diagnoses, illnesses, medical predictions, recommendations, etc. made by the expert engine 102 or the health predictor module 103 described in more detail below. Likewise, as indicated with respect to the configuration of FIG. 1, the database 106 may store time data for any and all of the parameters identified above, such as when the each measurement or input data was taken or received, the times associated with the data (if entered later than the time to which the data pertains) previous illness, diseases, symptoms, etc., times for meal and/or drug intake, etc. Moreover, it will be understood that the data stored in the database 106 can be data of various types, including quantitative data (e.g., temperature, blood pressure measurements, etc.) as well as qualitative (good/bad, pain level on a scale of one to ten, etc.)

Additionally, as illustrated in FIG. 10, the expert system 102 is connected to the processor or controller unit 108, both of which may use any of the data stored in the database 106 and/or provided by the input system 104. The expert system 102 may be any type of expert system including a rules based system, or a model based system. In particular, the expert system 102 may be implemented as a neural network system or using a neural network, a partial least squares (PLS) system, a model predictive control system, a principal component analysis system, a regression system, etc. In general, the expert system 102 may be a model based system that uses a trained model (such as a neural network model, an MPC model, a regression model, etc.), which operates on a set of training data, such as data stored in the database 106, to generate the model, and the model may be used thereafter to perform diagnosis based on new data input into and stored in the system 100. The model may be retrained from time to time using new or more recent data to update the model. Moreover, the expert engine 102 may use the model and data stored in the database 106 to perform diagnosis and, additionally, may use data derived from the stored or input data, including the trend data, baseline data, the last measured data, the median or mean (or other statistical) data, changes or deviations from the trends or baseline data, or data that has been generally accepted or regarded as normal parameters, etc., which may be computed by the controller 108 for any given time or time period. Generally speaking, the expert engine 102 may use one or more models that are created and stored in a model repository 109 to model the operation of the user's body based on the input data about the user and the user's environment, intake, outputs, etc.

Additionally, as shown in FIG. 10, the expert engine 102 may refine or tweak the analysis it performs using a feedback or update loop 130. In particular, the expert engine 102 may determine that better, more recent, or new types of data are needed to perform a more complete analysis or diagnosis, and may obtain this data via the update loop 130. In particular, the expert engine 102 may, when making a diagnosis, determine that certain input data is out of date, or is not available, and may use the update loop 130 to acquire this data and rerun or refine the analysis based on this new data. In particular, the update loop 130 may query the input system 104 to obtain more or refreshed data about any desired data input, such as a new cholesterol measurement, blood pressure measurement, etc. The update loop 130 may ask the user to input the requested data via one or more of the input devices 122, may engage an appropriate sensor 110 or 112 for the new data, may access a server or other external data storage device via the internet connection 116 or the phone connection 118 to obtain the required data (which may be ambient environment data, a new set of symptoms for illnesses, etc.) Moreover, the update loop 130 may include an observed conditions block 132 which may provide more information needed by the expert engine 102 on observed conditions. For example, the block 132 may determine from the user whether the user has or does not have one or more symptoms that may be needed by the expert engine 102 to further refine a diagnosis, but for which no or no recent data has been collected. The block 132 may, for example, query the user to answer one or more questions regarding the existence or non-existence of certain conditions (e.g., is the user experiencing night sweats, dry skin, headaches, etc.) The block 132 may instruct the user to take additional measurements or collect additional data, may have the user perform one or more actions and then take additional data, or may have the user perform a series of actions in a particular order to obtain new or updated data (such as to have the user take deep breaths, drink water, etc.) Of course, the block 132 may query the user via the user input device 104, or may obtain the data in other manners and provide the updated or new data back to the expert engine 102 for further use in performing further diagnoses.

Still further, as indicated above, the system 100 may include a health predictor module 103 which may be used to predict future heath conditions or issues. Unlike the expert engine 102 which is used to diagnose current health conditions, the block 103 may analyze the data within the database 106 to determine trends or cycles that may be used to predict future conditions. For example, the block 103 may include a data processing unit that processes the data within the database 106 to observe trends or cycles that indicate or that are related to health issues. For example, the data processing unit of the predictive block 103 may determine if there is a correlation between blood sugar levels and headaches for the user at one or more times in the future. In this example, the data processing unit of the predictive block 103 may process the personal health data stored in the database 106 to look for high or positive correlations between various different parameters at the same or at different times. The block 103 may, in this example, determine that a blood sugar level above a certain amount generally leads to a headache about 10 hours later. The data processing unit of the block 103, upon making this determination or recognizing this factor, may store a rule (or a model) in the database 109 to be used in the future to make health predictions. The a predictive unit of the block 103 may also use these rules to predict future health issues, such as detecting when the blood sugar level is above the particular range and telling the user that the user is likely to have a headache in about 10 hours based on the stored rule or model. The predictive block 103 may additionally use the data in the database 106 to recommend an action to prevent or minimize the health condition (such as telling the user to take a pain medication, vitamins, etc.) to reduce the condition or to minimize the likelihood of the condition actually arising. Of course, the block 103 may store the predictions and the recommended actions in the database 106 and further analyze that data, at a future time, to see whether the recommended action reduced or eliminated the problem or heath issue, and may use this further data in the next prediction and recommendation process.

Of course, the various portion of the block 103 may be executed by the processor or controller 108 in the background periodically or in a continuous manner to test for and determine correlations in the stored data to thereby generate predictive rules to be used to make predictions. To determine correlations, the data processing unit of the block 103 may select various different groups or types of data as stored in the database 106 to test for correlations, may select or use any different number or combinations of the data and may change the time lags or time cycles associated with the different types of data to determine potentially highly correlated data to be used to make predications. Moreover, the data processing unit of the block 103 may select or change the groups of data (the various different combinations of parameters) and/or the time lags between these data groups to use in the analysis in a systematic manner, in a random manner or in a semi-random manner. Still further, the data processing unit of the block 103 may change the periods of time over which the various data parameters are used (e.g., one day, one month, one hour, 5 minutes, etc.) and the amount of data that used in each correlation determination. The data processing unit of the block 103 may use the raw data, or may preprocess the raw data and operate on processed or statistical data, e.g., on means, medians, standard deviations, etc. of the stored data over various time periods. The block 103 may also operate on detected baselines, normal values, trends and deviations from these values. As will be understood, the block 103 may test combinations of or may combine data parameters in any manner (including physical parameters, ambient data parameters, food intake parameters, diagnostic parameters, etc.) with the analyses limited only by the amount of data present in the database 106 or accessible via the data input unit 104.

While the block 103 has been described as making predictions based on detected correlations in the data, the block 103 may determine or perform the correlation analysis in any desired manner. For example, the block 103 may use one or more data models (such as any of the models stored in the data repository 109), including a neural network model, a PLS model, an MPC model, or other data model, and may run a principal component analysis, a regression analysis, etc. in making the correlation determinations.

Generally speaking, as will be understood, both the expert engine 102 (in making diagnostic determinations) and the predictive module 103 (in making predictive analyses) are generating or are using one or more models (determined using the personal health data stored in the database 106) that model the reaction of or the operation of the user's body. Stated in another manner, these models are created to model or predict various cycles that exist in the user's body, such as blood sugar cycles, oxygen absorption cycles, food cycles, drug response cycles, etc. Such cycles may exist between any two or more of the parameters stored in the database 106 and between any parameters or groups of parameters and health issues or health conditions, and generally speaking, it is important to determine the parameters that are involved in a meaningful cycle and the most relevant time lag or time lags between these parameters that define a meaningful and correlated relationship between the parameters. Moreover, these models (or cycles) will change over time as the user's body changes (ages, is exposed to different environments, illnesses, etc., takes drugs, changes exercise or food intake habits, etc.) and so the models should be updated to reflect the changes in the user's body or environment. Moreover, the model or models generated and/or used by the expert engine 102 and the predictive module 103 are personal models that are specifically tailored to and reflect the particular user being modeled (and may differ significantly from user to user). These models are therefore more accurate and predictive for that user, especially as more and more data is collected for the user and the models are refined, tweaked, or regenerated based on the newly collected data.

In one case, the prediction module 103 may predict future health conditions from current conditions and trends. To do so, the prediction module 103 may look at time relationships of parameters (e.g., the relationship of exercise to a rise in body temperature), may compare current observed conditions (over time) with parameter changes (over time), may use trends of parameters and known relationships between parameters to predict future conditions (by, for example, creating and running models that encapsulate these relationships), etc. Moreover, the system 100 may use or allow the verbal entry of data rather than requiring manual entry of data via a keyboard, for example. Such a verbal entry may enable the user to easily enter the type and nature of food/drink consumed, the time and amount of the consumption, the waste discharged (and time and approximate amount), observed conditions in the form of, for example, weather, pain or aches being felt, when the user feels nauseous, etc.

As an example, the module 103 may measure or determine such things as glucose, blood pressure, oxygen saturation (O₂) which may be used to measure or detect respiratory disorders, cholesterol, weight, sleep, consumption, heart rate, body temperature, age, etc. As another example, the prediction module 103 may determine the relevant or most relevant time lags between certain data parameters and health events to determine or model body cycles. For example, the user may measure O₂ levels when enriched oxygen has been introduced into the user's body, and the module 103 may determine the time lag as to when the higher O₂ levels start to show up at extremities, such as at the user's fingers or toes. In this case, the module 103 may measure the amount of O₂ increase and the period of increase (i.e., the time lag between the increase and the introduction of O₂.) Here, the peak time may represent the O₂ cycle of the user's body. In other cases, the module 103 may measure the time lag between an increase in body temperature and heart rate, the time lag between cholesterol rising in the blood and the consumption of meals, the time lag between an increase or decrease in blood sugar levels and consumption of meals, the time lag between water consumption and temperature/O₂/heart rate/etc. The module 103 may also use the determined cause and effects or cycles to find relationships or to find causes of events. The module 103 may thus find the relationships between blood pressure and heart rate, blood flow, resistance, exercise, pulse rate, cholesterol, weight (gain or loss), gfr (glomerular filtration rate), body temperature, glucose tolerance, kidney disease (diagnosis), etc. Moreover, the module 103 may find the relationships between cholesterol levels (or changes thereof) and blood pressure, heart disease (e.g., if the user has been diagnosed with heart disease or has a family history of heart disease), triglycerides, diabetes (e.g., if the user has been diagnosed with heart disease or has a family history of heart disease), testosterone, estrogen, vitamin d intake, etc. The module 103 may find relationships between any and all of the collected data parameters, and the trends between these relationships over time could be important. Moreover, the module 103 may use identified relationships and/or trends to diagnose, predict, or create trends, to create an index of all known relationships as to the state of health, etc.

As another example, the module 103 may use leading indicators to perform health predictions. In particular the module 103 may search the database 106 or the parameters therein for health conditions (such as headaches, stomach aches, epileptic episodes, etc.) and find which data parameter or group of data parameters are correlated therewith in some manner, and the most relevant time delay associated with the correlations. The module 103 may, for example, run a cluster analysis and/or a multiple linear regression analysis on the data parameters with respect to the health event to determine the most relevant leading indicators (e.g., the parameters that are relevant or correlated to the health event, the data values of the relevant parameters, and the time lags associated with relevant parameters and the health event) to thereby determine one or more leading indicators of the health event, for the person. Thereafter, the module 103 may build a model that is used to look for these leading indicators, and to predict the health event in the future at a time based on or consistent with the determined time lags. Moreover, the outputs of the heath prediction module 103 may be used as inputs to the expert system 102 to cause the expert system 102 to perform a diagnosis based on the predicted conditions.

In still another case, the predictive module 103 may predict body parameters that generally need to be measured in a lab or using an external test (e.g., a blood sugar level). In this case, the module 103 may use a regression analysis, a principle component analysis, etc. to find a set of parameters (e.g., physical and/or environmental parameters stored in the database 106) that are most relevant to the lab measured values (assuming that the lab measured values are provided to the system 100 for the times relevant to the stored data). Thereafter, the module 103 may create a model that reflects these relationships or correlations, and may use the model to predict the parameter that can only be measured in the lab or via an external test, based on the recent set of physical and/or environmental data for which no test or lab measurement has been made. In this manner, the predictive module 103 may operate to predict a physical parameter that can only actually be measured in a lab or using an external test, and may provide that predicted value to the expert engine 102, may use the predicted value in other predictions or models, etc. Such a system may, for example, be used to predict blood sugar levels in a person (e.g., with diabetes) to lessen the number of blood tests the user has to perform on a day to day basis.

As illustrated in FIG. 10, the system 100 may include a recommendation block 150 that may use the diagnosis or future heath predictions output by the block 102 and 103, as well as data stored in the database 106 to recommend one or more actions to be taken by the user. The recommendation unit or block 150 may, for example, search the database 106 for therapies, drugs, or other recommendations to alleviate, minimize or treat a detected condition (diagnosis), a predicted condition, etc. The recommendation block 150 may take other information into account in making recommendations, such as other drugs that the user is taking, allergies, family history, or other physical conditions of the user (e.g., high blood pressure, high cholesterol, etc.) in selecting or choosing which recommendations to make to the user. Thus, for example, if the user is predicted to have a headache, or has body aches, but is allergic to ibuprofen, the recommendation unit 150 may recommend aspirin or Tylenol, but not ibuprofen, even though aspirin and Tylenol are generally less effective than ibuprofen. Moreover, the recommendation unit 150 may use relationships or rules generated by the predictive block 140 to make recommendations, e.g., if the user is more responsive to aspirin than ibuprofen, as detected in the past by the block 103, the recommendation unit 150 may recommend aspirin for a predicted headache.

As illustrated in FIG. 10, the system 100 also includes an output block 160 which may take the diagnosis output by the expert engine 102 or the predicted condition produced by the predictive block 103, and/or the recommendations provided by the recommendation unit 150 and perform some action based thereon. For example, the output block 160 may display or otherwise provide the prediction, diagnosis, or recommendation(s) to the user via an output device (e.g., a display screen, a text message, an e-mail, a voice system, etc.) Still further, as indicated above, the block 160 may provide the diagnosis or predicted health issue or even the recommended action to a doctor, therapist, pharmacy, etc. as set up or specified by the user. This information may be sent in any desired manner, such as via e-mail, a text message, a personal logon account with a doctor's office, etc. Still further, the output unit 160 may perform some actions related to the displayed or provided output, such as reminding the user to take prescribed drugs in a timely manner, to check blood sugar levels, to eat or drink something to prevent the onset of a diabetic episode, etc. Generally speaking, it is desirable for the output unit 160 to, in some cases, provide graphs, charts, or plots of relevant parameters that illustrate or that are associated with the diagnosis or the predicted health issue. Such plots may, for example, illustrate the relationship between two or more parameters, the normal regions and the current values for one or more data parameters relevant to a health issue, or any other types of plots or graphs that make the data relevant to the condition most understandable. These plot or graphs may take the form of, for example, parallel plots, spider plots, cluster plots, etc., although other types of graphs, plots and charts could be used. Such plots and charts are useful to be provided to the doctor to enable the doctor to quickly understand the reasoning behind the analysis, prediction or diagnosis determined by the system 100.

It will be noted that the system 100 may also include a data processor or cleaner 170 which is used to clean, filter, preprocess, etc. the data collected and stored in the database 106. In particular, it is very important to clean the data used by the predictive module 103 and in the expert engine 102 to assure accuracy of the predictions and diagnoses. The data cleaner 170 may use any of various known techniques to clean the data, including filtering the data, removing outlier data, analyzing the data to assure it is likely to be accurate etc. In particular, the data cleaner 170 may analyze the data to see if the data is all the same, has similar or repetitive patterns, etc., any of which may indicate that the data is not as accurate is it could be. In particular, in some cases, such as when a user is asked to input data manually, it is possible that the user may make up data or try to remember data. In many cases, when doing this, the user may enter the same number for the data (even though that number is not accurate), may repeat a pattern of data, etc. These patterns may indicate that the data is not reliable enough to use to make predictions or to detect patterns or correlations, and so the data cleaner 170 may eliminate this data from consideration by the units 102, 103, 150, etc. Still further, the data cleaner 170 may analyze time stamps associated with when the data was first stored in the database 106 to determine if the data is input relatively simultaneously or contemporaneously with the time to which the data is related, or if the data is entered much later (indicating that the data may not be as reliable). Still further, the data cleaner 170 may recognize data streams that are missing enough data measurements to be unreliable and may eliminate this data or mark this data as being unreliable or suspect for use in the predictive or diagnostic analyses. Still further, the data cleaner 170 may fill in missing data using extrapolation (based on a line or a curve of some sort or using any known extrapolation algorithm) or using interpolation. Likewise, the data cleaner 170 may, using some or all of the factors stated above, as well as the source of the data (e.g., whether the data comes from a sensor or is input manually by a user), assign a reliability factor to the data. Thereafter, the predictive unit 103 and the expert engine 102 may use the reliability factor to assess or estimate the reliability of the diagnosis or prediction, to determine what data to use in the prediction or correlation analysis or diagnosis, etc.

While the personal health monitoring system 100 has been described herein as a stand-alone unit incorporated into a phone, tablet or other personal computing device, some or most of the features of the personal health monitoring system 100 described herein can be implemented in a distributed manner, such as in a server (or in the cloud) in conjunction with a personal computing device. For example, the input and display features described above may be implemented in a personal computing device, such as a phone or a tablet computer, while any or all of the predictive module 103, the expert engine 102, the database 106, the recommendation unit 150 and the data cleaner 170 can be implemented in one or more servers or other computing devices connected to the personal computing device via a wired or a wireless connection. Generally speaking, these features, which are typically more computationally expensive or memory intensive, can be implemented in a higher power processor/memory within a server, which can communicate with the personal computing device to access or acquire new data, and to provide outputs (e.g., recommendations) to the user. In this case, the personal computing device and the server or servers will communicate via a communication network using standard or known communication interfaces.

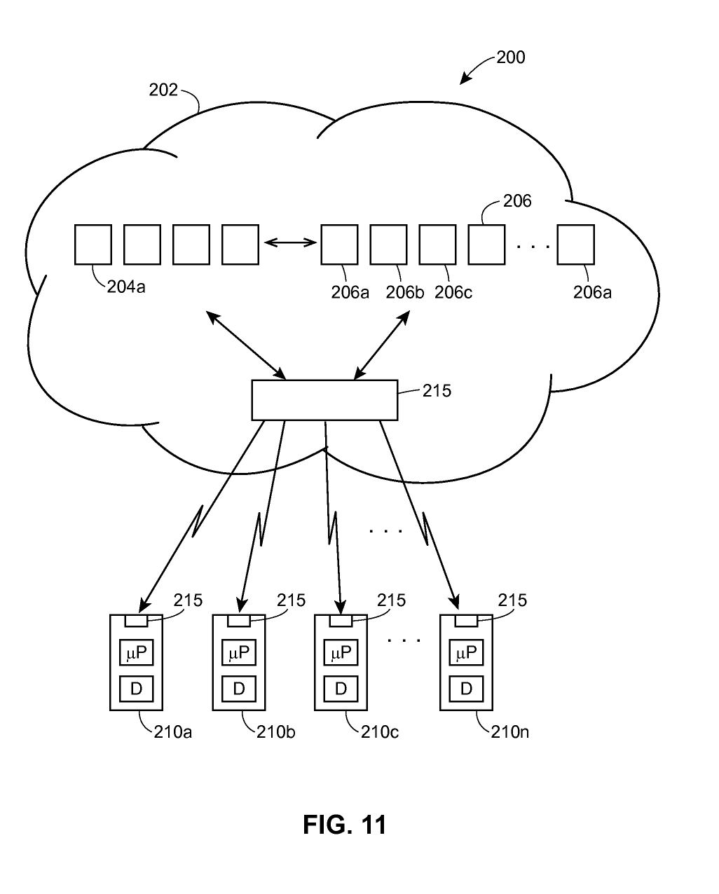

FIG. 11 illustrates a cloud based personal health monitoring system 200 that communicates with and supports multiple different people or personal health monitors. In particular, the system 200 of FIG. 11 includes a cloud (or otherwise remotely based) server or server network 202 including processors 204 and data storage units 206. The server or server network 202 stores personal heath monitoring data (e.g., any or all of the data indicated above as being collected by, generated by or stored by the personal health monitoring systems of FIGS. 1-10) for each of multiple different people or users. Likewise, the server network 202 can store and implement (execute) on the processors 204 any or all of the various different diagnostic, predictive, recommendation and data cleaning modules described above with respect to FIGS. 1-10, including the expert engine 102, the predictive module 103, the recommendation module 150, etc. These resources may be shared by various users having personal devices 210a-210n to perform personal health monitoring for each of the users, based on those users' personal data stored in the databases 206a-206n. The users 210a-210n may each have a personal device, such as a phone, a laptop computer, etc., which includes input mechanisms, such as some are all of the input devices associated with the input unit 104 of FIG. 10, and may include any number of output devices such as display devices, text messaging or e-mail routines, voice generation devices, alarm devices, etc. The devices 210a-210n communicate, preferably wirelessly, with the server network 202 via communication interfaces 215 located in the devices 210a-210n and the server network 202. The communication interfaces 215 may implement any desired type of communications using any desired or known communication protocol, including HTTP, internet based protocols, cellular data protocols, etc.

While the system 200 of FIG. 11 illustrates that most of the personal health monitoring processing is performed at the server network 202, some or all of this processing could be implemented in the devices 210a-210n (each of which includes a processor) to perform for example, data collection, data cleaning, predictive analysis, diagnostic analysis, etc. Moreover, different parts of these types of data processing could be split in different manners for different ones of the devices 210a-210n, depending on the processing power and memory capabilities of the devices 210a-210n, user preferences, etc. Likewise, if desired, a user could store his or her personal data in one of the devices 210a-210n and send that data to the server network 202 for processing when desired, to thereby keep the personal data more confidential (as it will not be stored permanently at the server network 202).

Importantly, using the system of FIG. 11, the diagnostic and predictive capabilities of the entire personal health monitoring system 200 can be improved over that of a single system, such as that of FIG. 10, by performing diagnostics, rule or correlation detection, and predictive analysis feedback using data from multiple people. That is, the predictive and correlation analysis routines can scan the time series data for multiple people looking for trends, baselines, correlation, time delays, etc., and can generate rules or predictive correlations based on a combination of this data or using data from multiple people. In some instances, the data from a single person may not be comprehensive enough to determine or detect a particular relationship or correlation between parameter values and predictive health issues or diagnostic conditions. However, analyzing the data from multiple people may provide more comprehensive data upon which to detect such correlations. Moreover, the correlations detected for one person and the rules or predictive results determined therefrom may be used for other people, or may be tested on the data for other people to determine if these rules, relationships, correlations and/or time delays are applicable more generally (i.e., are applicable to other people). As a result, the availability of more data for multiple persons enables the predictive and diagnostic routines at the server network 202 to be more accurate, to detect rules or correlations that are applicable to multiple people, to determine or narrow down time relationships between various inputs and output parameters (health issues), etc.

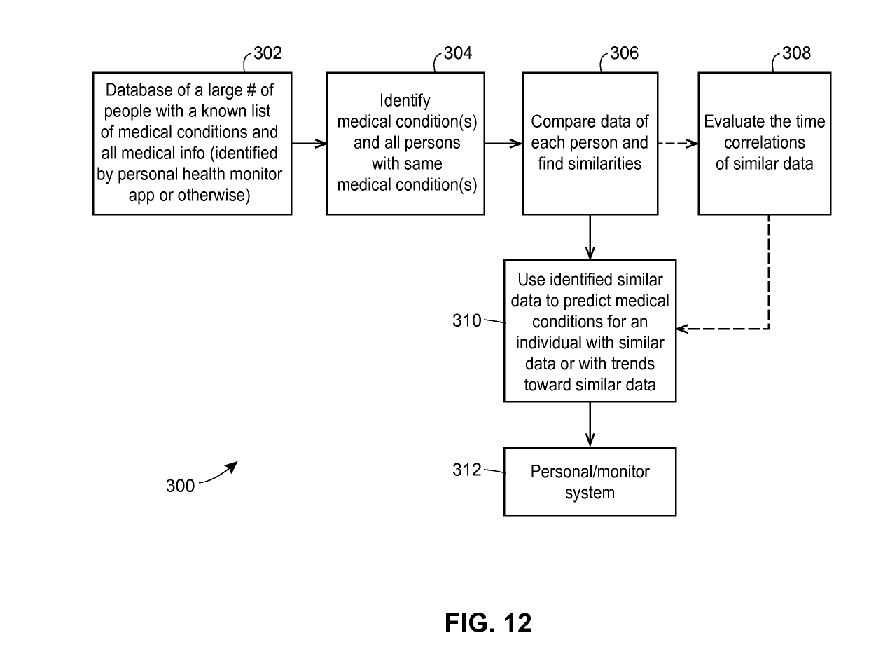

FIG. 12 illustrates a routine 300 that may be used at, for example, the server network 202 of FIG. 11 to perform diagnostic and predictive detections at a server based on data from multiple different people. In particular, a block 302 stores a database of a large number of people with a known medical condition or a known set of medical conditions and the medical data, personal data, environmental data, etc., for each of those persons. The known medical conditions may be identified by the user themselves (e.g., a self-reported condition) or may be conditions determined by or predicted by the personal health monitoring systems of those persons. Essentially, the block 302 stores the all of the data for a group of people who each have a personal health monitoring system. However, it is possible that some of the people in the database 302 may not have a personal health monitoring system but that, instead, the data for these people comes from other sources.

At a block 304, a routine, such as an expert engine or other condition or predictive model searching engine, identifies or selects one medical condition or a combination of medical conditions to analyze. Moreover, the block 304 then identifies the persons (or the data for the persons) who have the selected medical condition or combination of medical conditions. Next, a block 306 compares or analyzes the data of each of the persons identified in the block 304 to find similarities in the data, including similar correlations, time delays, ranges, etc. A block 308 may be used to analyze or detect particular time correlations between sets of data to determine a typical value for or a range of time delays at which the data from different parameters is correlated with respect to the one or more medical conditions. The output of the blocks 306 and 308 may be stored as rules, models, or relationships (correlations) to be used in predictive or diagnostic analyses by other routines.

Thereafter, as indicated by a block 310, a predictive routine either at the server network 202 or at an individual personal health monitoring device 210 may search a person's data (for a person who is not known to have the medical condition or the combination of medical conditions) to determine if the relationships or correlations (including time delays) between various data parameters matches or conforms with the relationships, correlations, and time delays identified by the blocks 306 and 308. If so, the block 310 may determine that the person may have the medical condition or the combination of medical conditions. Thereafter, a block 312 may send a message to the personal health monitoring system or device 210 of that person to inform the person of the potential condition or combination of conditions.

Of course, the system 200 of FIG. 11 can analyze and process the data from multiple persons in many other manners to identify generally applicable parameter and time delay relationships to be used to perform diagnostics and predictions regarding a person's health or health conditions.

Still further, it will be appreciated that the personal health monitoring systems described herein can be advantageously used to assist doctors in diagnosing patient issues or health concerns, in testing the effectiveness of pharmaceuticals, in providing on-going care or treatment, etc. In some cases, for example, a doctor may provide a kit to a user including the personal health monitoring system described herein to have the patient collect data and to receive preliminary diagnosis therefrom to help or assist the doctor in diagnosing a patient. Likewise, the personal health monitoring system can give a doctor updated and real-time information as the efficacy of a drug or treatment regime to better enable the doctor to diagnose an unknown condition or to treat a known health condition.

While the invention has been particularly shown and described with reference to a preferred embodiment, it will be understood by those skilled in the art that various changes in form and detail may be made therein without departing from the spirit and scope of the invention. For example, it would be understood by those skilled in the art that accumulating large amounts of data from an ever increasing list of individual parameters that can be measured over time could result in a significant improvement in the results of any analysis. Further it should be appreciated by one skilled in the art that the flow diagrams shown in FIGS. 9A, 9B and 12 are only illustrative of determining a user's health. There are a host of mathematical tools available that could be used in analyzing the data such as principal component analysis which could be performed on the data to identify contributing parameters (attributes) that resulted in certain illnesses. As one skilled in the art would appreciate, principal component analysis can be used to take a look at the raw data from various parameters to determine what the important contributors were that caused that particular result. This analysis can be running in the background of a processor while data is being collected and called upon by the user. Once the important contributing parameters are identified, the principal component analysis can then be directed to the finer or more limited set of parameters to validate the analysis. In situations where the system has sufficient data with regard to a user, the system can start with the problem, disease, illness, or aliment and then, using this analysis, look for contributing causes. As one skilled in the art would appreciate, there are many more mathematical tools available that can similarly be adapted and used on the collected data to discover root causes and effects of various conditions of the user. For example, various mathematical models are currently being used in the process control industry and can similarly be adapted and used to predict the oncoming of certain conditions such as cold sores, colds, heart disease, diabetes, etc. Still further, it should be appreciated to those skilled in the art that this personal health monitoring system could be used to monitor the health and well-being of pets or domestic animals.

What is claimed:

1. A method for monitoring the well-being of an individual, comprising:
collecting data from at least one sensor detecting physical attributes of an individual in memory of a mobile computing device, wherein the data represents the physical attributes;
implementing a model-based expert engine to analyze the data representing the physical attributes to determine a medical diagnosis related to the individual; and
providing the medical diagnosis via a user interface.

2. The method of claim 1, wherein implementing the model-based expert engine includes using a processor to train a model of the model-based expert engine on a portion of the collected data.

3. The method of claim 1, wherein implementing the model-based expert engine includes using one of a neural-network model, a model predictive control model, a partial least squares model or a regression model within the expert engine.

4. The method of claim 1, further including cleaning the data representing the physical attributes prior to using the data representing the physical attributes in the expert engine.

5. The method of claim 1, further including using the data representing the physical attributes to determine a model for one or more body cycles within the individual.

6. The method of claim 5, further including using the model on a processor to predict a future health condition of the individual.

7. The method of claim 1, further including using a processor to determine correlations between various different physical attributes and health conditions based on the data representing the physical attributes and using the correlations to predict a future health condition of the individual.

8. The method of claim 7, wherein determining correlations between various different physical attributes and health conditions includes determining one or more time delays associated with the correlations.

9. The method of claim 1, wherein implementing the expert engine includes performing a feedback loop in which the expert engine obtains further data representing one or more a physical attributes or health conditions to determine the medical diagnosis.

10. A personal health monitoring device, comprising:
a memory module capable of storing data representing multiple attributes of an individual, wherein the data has a time parameter associated with it to identify a time associated with the data;
a processor;
a data processing module stored in the memory module and operable on the processor to detect correlations between various different physical attributes and health conditions based on the data representing the multiple attributes of the individual;
a prediction module stored in the memory and operable on the processor to use the detected correlations between various different physical attributes and health conditions to determine a health diagnosis or to predict a future health condition of the individual; and
an output device that provides an indication of the diagnosis or the predicted future health condition of the individual.

11. The personal health monitoring system of claim 10, wherein the prediction module includes an expert engine that uses a model to determine a health diagnosis for the individual and wherein the data processing module develops the model for use by an expert engine.

12. The personal health monitoring system of claim 11, wherein the data processing module operates on the processor to train the model of the expert engine on a portion of the stored data.

13. The personal health monitoring system of claim 10, further including a data cleaning module that cleans the stored data prior to the data processing module using the stored data.

14. The personal health monitoring system of claim 10, wherein the data processing module uses the stored data to determine a model for one or more body cycles within the individual.

15. The personal health monitoring system of claim 14, wherein the prediction module uses the model to predict a future health condition of the individual.

16. The personal health monitoring system of claim 10, wherein the data processing module operates on the processor to determine correlations between various different physical attributes and health conditions within the stored data and wherein the prediction module uses the correlations to predict a future health condition of the individual.

17. The personal health monitoring system of claim 16, wherein the data processing module determines one or more time delays associated with the correlations.

18. A method for monitoring the well-being of an individual, comprising:
collecting data from at least one sensor detecting physical attributes of an individual;
storing the data for the physical attributes of the individual in a memory of a computing device;
implementing a data processing module on a processor to analyze the data representing the physical attributes to determine one or more correlations between various different physical attributes and health conditions based on the data representing the physical attributes;
using the correlations to predict a future health condition of the individual; and
providing an indication of the predicted future health condition of the individual via a user interface.

19. The method of claim 18, wherein implementing the data processing module to determine the correlations includes using the data representing the physical attributes to determine a model for one or more body cycles within the individual.

20. The method of claim 18, wherein determining the correlations between various different physical attributes and health conditions includes determining one or more time delays associated with the correlations.
